(19) United States
(12) Patent Application Publication
(10) Pub. No.: US 2018/0116921 A1
(43) Pub. Date: May 3, 2018

(54) ANTIPERSPIRANT COMPOSITION WITH IMPROVED SKIN FEELING

(71) Applicant: Henkel AG & Co. KGaA, Duesseldorf (DE)

(72) Inventors: Thomas Doering, Dormagen (DE); Natascha Schevardo, Erkrath (DE)

(73) Assignee: Henkel AG & Co. KGaA, Duesseldorf (DE)

(21) Appl. No.: 15/784,208

(22) Filed: Oct. 16, 2017

(30) Foreign Application Priority Data
Nov. 3, 2016 (DE) 10 2016 221 536.1

(51) Int. Cl. A61K 8/26; A61Q 15/00; A61K 8/60; A61K 8/33; A61K 8/37; A61K 8/28

(52) U.S. Cl. CPC A61K 8/26; A61Q 15/00; A61K 8/28; A61K 8/33; A61K 8/375; A61K 8/60

(57) ABSTRACT

The present invention concerns antiperspirant agents which contain, in an aqueous or aqueous-alcoholic carrier, antiperspirant aluminum salts in combination with a selected alkyl(oligo)glucoside, a selected dialkylether and a glycerine ester. The antiperspirant agents have improved cosmetic features, more particularly a reduced sticky effect on the skin.

The present invention concerns an antiperspirant agent containing, in an aqueous or aqueous-alcoholic carrier, at least one antiperspirant aluminum salt, as well as the use thereof in a non-therapeutic, cosmetic process for preventing and/or reducing body transpiration.

Washing, cleansing and caring for one's own body are a basic human need and modern industry continuously attempts to meet these human needs in a variety of ways. Long-lasting elimination or at least reduction of the body odor and underarm wetness are especially important for daily hygiene. Numerous deodorizing or antiperspirant personal care products, which were developed for use in body regions with a high density of sweat glands, more particularly in the underarm region, are known from the prior art said agents are packaged in a wide variety of dosage forms, such as a powder, stick, aerosol spray, pump spray, liquid and gel-like roll-on application, lotion, gel and as a moist flexible substrate (deodorant wipes). Packaging the antiperspirants in liquid form has advantages with respect to dosage and application, by means of an atomizer, roll-on or spray, for example.

Cosmetic antiperspirants from the prior art contain at least one antiperspirant salt. An aluminum salt, such as aluminum(III)-chloride or an aluminum zirconium salt, for example, is used as the antiperspirant salt. The use of aluminum salts or aluminum zirconium salts in the quantities required to achieve an adequate antiperspirant effect of usually more than 8 wt. %, relative to the total weight of the agent, leads to a poor tolerance of the skin towards said agent. The use of increased quantities of these antiperspirants also severely impacts the cosmetic properties, more particularly a long-lasting sticky sensation on the skin.

For this reason, the prior art has attempted to increase the efficiency of aluminum and aluminum zirconium salts by means of activation, in order to review the total quantity of used antiperspirant agent and thereby improve the cosmetic properties.

However, activated aluminum and aluminum zirconium salts continue to have the disadvantage that they reduce the antiperspirant effect as the storage period increases and/or if protic solvents are used. This is because the salts, more particularly the halides, tend to form in aqueous solutions and in the normally used pH range from pH 4 to pH 7, polymeric aluminum or zirconium complexes with hydroxide or oxide bridges, which have a high molecular weight and a low antiperspirant effect.

There is therefore a need for aqueous or aqueous-alcoholic antiperspirant agents, which do not significantly reduce the antiperspirant effect even when stored for prolonged periods and/or if high quantities of protic solvents are used. Moreover, the antiperspirants ought to have good cosmetic properties, more particularly a reduced sticky effect on the skin and good skin tolerance.

This problem is solved by means of an antiperspirant agent containing, in an aqueous or aqueous-alcoholic carrier

a) at least one antiperspirant aluminum salt, selected from the group consisting of water-soluble inorganic aluminum salts, water-soluble inorganic aluminum salts, water-soluble inorganic aluminum zirconium salts, water-soluble organic aluminum zirconium salts and the mixtures thereof,

b) at least one alkyl(oligo)glucoside of the general formula RO-[G]ₓ, wherein R denotes an alkyl- and/or alkenyl radical having 4 to 22 carbon atoms, G denotes a sugar radical having 5 or 6 carbon atoms and x denotes integers from 1 to 10,

c) at least one dialkyl ether with the same or different C4- to C14 alkyl radicals and

d) at least one glycerine ester having a fatty acid, selected from the group consisting of fatty acid monoglycerides, fatty acid diglycerides and the mixtures thereof.

It has now unexpectedly emerged that the combination of certain antiperspirant compounds with selected alkyl(oligo)glucosides, selected dialkyl ethers and a glycerine ester in an antiperspirant agent with an aqueous or aqueous-alcohol carrier leads to improved cosmetic properties, more particularly to a reduced sticky effect on the skin.

The term “antiperspirant” means the reduction of transpiration of the sweat glands of the body.

As a first essential constituent (a), the antiperspirant agent contains at least one antiperspirant aluminum salt.

According to the present invention, the at least one antiperspirant aluminum salt is selected from the group consisting of

(i) water-soluble inorganic salts of the aluminum, more particularly aluminum chlorhydrate, aluminum sesquichlorhydrate, aluminum dichlorhydrate, aluminum hydroxide, potassium aluminum sulfate, aluminum bromhydrate, aluminum chloride, aluminum sulfate;

(ii) water-soluble organic salts of of the aluminum, more particularly particularly aluminum chlorohydrex propylene glycol, aluminum chlorohydrex polyethylene glycol, aluminum propylene glycol complex, aluminum sesquichlorohydrex propylene glycol, aluminum sesquichlorohydrex polyethylene glycol, aluminum propylene glycol dichlorohydrex, aluminum polyethylene glycol dichlorohydrex, aluminum undecylenoyl collagen amino acid, sodium aluminum lactate, sodium aluminum hydroxy lactate, aluminum lipo amino acids, aluminum lactate, aluminum chlorohydroxyallantoinate, sodium aluminum chlorohydroxy lactate;

(iii) water-soluble inorganic aluminum zirconium salts; more particularly aluminum zirconium trichlorohydrate, aluminum zirconium tetrachlorohydrate, aluminum zirconium pentachlorohydrate, aluminum zirconium octachlorohydrate;

(iv) water-soluble organic aluminum zirconium salts, more particularly aluminum zirconium propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine; as well as

(v) the mixtures thereof.

The at least one antiperspirant aluminum salt is preferably contained in the antiperspirant agent in a ratio from 5 to 25 wt. %, more preferably from 6 to 20 wt. %, even more preferably from 7 to 15 wt. % and most preferably from 8 to 14 wt. %, each of the quantity values referring to the total weight of the antiperspirant agent.

In a particularly preferred embodiment, the at least one antiperspirant aluminum salt is selected from the group consisting of aluminum chlorhydrate, aluminum sesquichlorhydrate, aluminum zirconium tetrachlorhydrexglycine and the mixtures thereof.

The expression “antiperspirant aluminum salts” does not include alumosilicates or zeolithes. Moreover, water-soluble aluminum salts are those salts which have a solubility of at least 3 wt. % at 20° C., i.e. at least 3 g of the antiperspirant aluminum salt dissolve in 97 g of water at 20° C.

Particularly preferred inorganic aluminum salts are selected from aluminum chlorhydrate, more particularly aluminum chlorhydrate having the general formula [Al2(OH)₅Cl.1-6 H₂O]ₙ, preferably [Al₂(OH)₅Cl.2-3 H₂O]ₙ, which can exist in a non-activated (polymerized) or in an activated (depolymerized) form, as well as aluminum chlorhydrate having the general formula [Al₂(OH)₄Cl₂.1-6 H₂O]ₙ, preferably [Al₂(OH)₄Cl₂.2-3 H₂O]ₙ, which can exist in a non-activated (polymerized) or in an activated (depolymerized) form.

The aluminum chlorhydrate is preferably contained in the antiperspirant agent in a ratio from 5 to 25 wt. %, more preferably from 6 to 20 wt. %, even more preferably from 7 to 15 wt. % and most preferably from 8 to 14 wt. %, each of the quantity values referring to the total weight of the antiperspirant agent.

Regular aluminum chlorhydrate usually has a molar Al/Cl ratio of approximately 1.9:1 to 2.1:1. Aluminum sesquichlorhydrate has a low molar Al/Cl ratio, often of approximately 1.5:1 to 1.8:1. Aluminum sesquichlorhydrate reacts in an aqueous solution more acidic than aluminum chlorhydrate.

The aluminum sesquichlorhydrate is preferably contained in the antiperspirant agent in a ratio from 5 to 25 wt. %, more preferably from 6 to 20 wt. %, even more preferably from 8 to 14 wt. % and most preferably from 10 to 12.5 wt. %, each of the quantity values referring to the total weight of the antiperspirant agent.

Other preferably usable aluminum salts are aluminum zirconium salts, which additionally contain the amino acid glycine. These antiperspirant aluminum salts are also referred to as

“ZAG compounds”. Preferred embodiments of the antiperspirant agents are therefore characterized in that the at least one antiperspirant aluminum salt contains an aluminum zirconium salt additionally containing glycine in a total quantity from 2.0 to 8.0 wt. %, preferably from 3.0 to 7.0 wt. %, more preferably from 3.5 to 6.5 wt. %, and most preferably from 4.0 to 6.0 wt. %, relative to the total weight of the antiperspirant aluminum zirconium salt.

In this context, it is also preferable for the molar ratio of glycine to zirconium in the aforementioned salts to be 0.5:1 to 1.5:1.

The antiperspirant agent can contain one or more antiperspirant aluminum salts.

The total quantity of antiperspirant aluminum salts is preferably 5 to 25 wt. %, more preferably 6 to 15 wt. % and most preferably 8 to 14 wt. %, relative to the total weight of the antiperspirant agent in each case.

As a second essential constituent (b), the antiperspirant agent contains at least one alkyl(oligo)glucoside of the general formula RO-[G]ₓ, wherein R denotes an alkyl- and/or alkenyl radical having 4 to 22 carbon atoms, G denotes a sugar radical having 5 or 6 carbon atoms and x denotes integers from 1 to 10,

Particularly suitable alkyl(oligo)glycosides (b) are derived from aldoses and/or ketoses having 5 or 6 carbon atoms, preferably from glucose. The radical R more preferably denotes an alkyl radical having 6 to 20 and most preferably 8 to 18 carbon atoms.

In the general formula RO-[G]ₓ, the index value x denotes the oligomerization degree (DP), i.e. for the distribution of mono and oligoglycosides. The index value x preferably has a value in the range from 1 to 6, more preferably in the range from 1 to 3, wherein said value is not an integer but can be a fraction, which can be determined analytically.

Particularly preferred alkyl(oligo)glycosides have an oligomerization degree of between 1.2 and 1.5.

Most preferred alkyl(oligo)glycosides are available in the trade from various suppliers under the INCI trade name “Decyl Glucoside”, “Lauryl Glucoside” and “Coco Glucoside”.

The alkyl(oligo)glycoside is used in the antiperspirant agents in quantities from 0.01 to 5 wt. %, more preferably from 0.02 to 2.5 wt. % and most preferably from 0.05 to 1 wt. %, relative to the total agent. Preferably, 0.01 to 5 wt. %, more preferably 0.02 to 2.5 wt. % and most preferably 0.05 to 1 wt. %, relative to the total agent, of an alkyl(oligo)glycoside, which is known under the INCI trade name “Decyl Glucoside”, is used in the antiperspirant agent as the alkyl(oligo)glycoside (b). “Decyl Glucoside” can be obtained from BASF SE under the trade name of Plantacare® 2000, for example.

As a third essential constituent (c), the antiperspirant agent contains a dialkyl ether with the same or different C4- to C14-alkyl radicals. Usable dialkylethers are particularly di-n-alkylether, such as for example di-n-octylether, di-n-decylether, di-n-nonylether, di-n-undecylether, di-n-dodecylether, n-hexyl-n-octylether, n-octyl-n-decylether, n-decyl-n-undecylether, n-undecyl-n-dodecylether and n-hexyl-n-undecylether, as well as di-tert.-butylether, di-iso-pentylether, di-3-ethyldecylether, tert.-butyl-n-octylether, iso-pentyl-n-octylether and 2-methylpentyl-n-octylether.

The di-n-octylether (dicaprylyl ether), commercially available under the trade name Cetiol® OE, is particularly preferred.

The dialkyl ether is used in the antiperspirant agents in quantities from 0.01 to 5 wt. %, more preferably from 0.02 to 2.5 wt. % and most preferably from 0.05 to 1 wt. %, relative to the total agent. Di-n-octyl ether is used in the antiperspirant agents as a dialkyl ether (c) in quantities from 0.01 to 5 wt. %, more preferably from 0.02 to 2.5 wt. % and most preferably from 0.05 to 1 wt. %, relative to the total agent.

As a fourth essential constituent (d), the antiperspirant agent contains a glycerine ester having a fatty acid selected from the group consisting of fatty acid monoglycerides, fatty acid diglycerides and mixtures thereof.

The fatty acids preferably have 12 to 30 carbon atoms, more preferably from 12 to 22 carbon atoms.

A C₁₂-C₃₀ fatty acid monoglyceride is the monoester of the trivalent alcohol glycerine with an equivalent of a C₁₂-C₃₀ fatty acid. Either the middle hydroxy group of the glycerine or a final hydroxy group of the glycerin can be esterified with the fatty acid.

A C₁₂-C₃₀ fatty acid diglyceride is the diester of the trivalent alcohol glycerine with two equivalents of a C₁₂-C₃₀ fatty acid. Either the middle and an independent hydroxy group of the glycerine with two equivalent fatty acids can be esterified with two equivalent fatty acids or both final hydroxy groups of the glycerin are each esterified with one fatty acid. The glycerin can be esterified with two identically structured or two different fatty acids.

The fatty acids are preferably selected from dodecan acid (lauric acid), tetradecan acid (myristine acid), hexadecanic acid (palmitic acid), tetracosanic acid (lignoceric acid), octadecanic acid (stearic acid), eicosanic acid (arachidic acid), docosanic acid (behenic acid), petroselin acid [(Z)-6-octadecenoic acid], palmitoleic acid [(9Z)-hexadec-9-enic acid], oleic acid [(9Z)-octadec-9-enic acid], elaidinic acid [(9E)-octadec-9-enic acid], eruca acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid].

The use of a fatty acid monoglyceride, more particularly of a fatty acid monoglyceride with linear C12-C22 fatty acids is particularly preferred. Examples of particularly suitable fatty acid monoglycerides are glycerolmonolaurate, glycerolmonomyristate, glycerolmonopalmitate, glycerolmonostearate or glycerolmonooleate. Glycerolmonooleate is particularly suitable.

Suitable diesters of the glycerine are those with linear fatty acids having 12 to 22 carbon atoms. Examples of particularly suitable glycerine esters are glyceroldilaurate, glyceroldimyristate, glycerlydipalmitate, glyceroldistearate or glyceroldioleate.

The usage quantity of the glycerine ester is usually 0.01 to 5 wt. %, more preferably 0.02 to 2.5 wt. % and most preferably 0.05 to 1 wt. %, relative to the total agent. Glycerinmonooleate is used in the antiperspirant agents as a glycerine ester having a fatty acid (d) in quantities from 0.01 to 5 wt. %, more preferably from 0.02 to 2.5 wt. % and most preferably from 0.05 to 1 wt. %, relative to the total agent.

The alkyl(oligo)glucoside, the dialkyl ether and the glycerine ester can be used separately as individual substances or as a pre-mixture, as an aqueous emulsion, for example.

The antiperspirant agent has an aqueous or an aqueous-alcoholic carrier.

Preferred antiperspirant agents exist as aqueous emulsions. Preferred cosmetic agents in the form of emulsions are characterized by a water content from 55 to 90 wt. %, preferably from 60 to 85 wt. % and more preferably from 65 to 80 wt. % (relative to the total weight of the agent in each case).

Alternatively, the antiperspirant agents exist as aqueous-alcoholic compositions, preferably as aqueous-alcoholic solutions.

To improve the application properties, it is preferable for the antiperspirant agents to contain, relative to their total weight, ethanol in quantities between 10 and 60 wt. %, preferably between 20 and 50 wt. % and most preferably between 30 and 40 wt. %.

In addition to the essential components, the antiperspirant agent can contain at least one further ingredient. The further ingredients can be selected from the group of (i) further emulsifiers and/or tensides; (ii) thickening agents; (iii) deodorant active ingredients with anti-bacterial and/or bacteriostatic and/or enzyme-inhibiting and/or odor-neutralizing and/or odor-absorbing effect; (iv) polyvalent alcohols and/or polyethylene glycols; (v) skin-cooling active ingredients; (vi) pH set-up agents; (vii) skin-nourishing active ingredients, such as skin-moistening substances, skin-calming substances, skin-brightening substances and/or skin-smoothing substances; (viii) preserving agents, (ix) chelating agents, as well as (x) the mixtures thereof.

The antiperspirant cosmetic agent can be applied by means of various methods. However, it is preferable for the antiperspirant agent to be applied as a roll-on. Application can be achieved by means of a roller ball applicator, for example.

The table below shows most preferred embodiments SM 1 to SM 24 of the cosmetic agent according to the invention (all values in wt. %, unless otherwise stated):

| | SM 1 | SM 2 | SM 3 | SM4 |
|---|---|---|---|---|
| Antiperspirant aluminum | 5-25 | 6-20 | 7-15 | 8-14 |
| salt | | | | |
| Alkyl(oligo)glucoside | 0.01-5 | 0.02-2.5 | 0.05-1 | 0.05-1 |
| Dialkyl ether | 0.01-5 | 0.02-2.5 | 0.05-1 | 0.05-1 |
| Fatty acid mono- or - | 0.01-5 | 0.02-2.5 | 0.05-1 | 0.05-1 |
| diglyceride | | | | |
| Water and/or ethanol | Ad 100 | Ad 100 | Ad 100 | Ad 100 |
| | SM 5 | SM 6 | SM 7 | SM 8 |
| Aluminum chlorhydrate | 5-25 | 6-20 | 7-15 | 8-14 |
| and/or aluminum | | | | |
| sesquichlorhydrate | | | | |
| and/or aluminum- | | | | |
| zirconium-glycine | | | | |
| compound | | | | |
| Alkyl(oligo)glucoside | 0.01-5 | 0.02-2.5 | 0.05-1 | 0.05-1 |
| Di-n-alkylether | 0.01-5 | 0.02-2.5 | 0.05-1 | 0.05-1 |
| C₁₂-C₂₀ fatty | 0.01-5 | 0.02-2.5 | 0.05-1 | 0.05-1 |
| monoglyceride | | | | |
| Water and/or ethanol | Ad 100 | Ad 100 | Ad 100 | Ad 100 |
| | SM 9 | SM 10 | SM 11 | SM 12 |
| Aluminum chlorhydrate | 5-25 | 6-20 | 7-15 | 8-14 |
| and/or aluminum | | | | |
| sesquichlorhydrate | | | | |
| and/or aluminum- | | | | |
| zirconium-glycine | | | | |
| compound | | | | |
| Alkyl(oligo)glucoside | 0.01-5 | 0.02-2.5 | 0.05-1 | 0.05-1 |
| with the INCI trade | | | | |
| name “Decyl Glucoside” | | | | |
| Di-n-octylether | 0.01-5 | 0.02-2.5 | 0.05-1 | 0.05-1 |
| Glycerolmonooleate | 0.01-5 | 0.02-2.5 | 0.05-1 | 0.05-1 |
| Water and/or ethanol | Ad 100 | Ad 100 | Ad 100 | Ad 100 |
| | SM 13 | SM 14 | SM 15 | SM 16 |
| Aluminum chlorhydrate | 5-25 | 6-20 | 7-15 | 8-14 |
| and/or aluminum | | | | |
| sesquichlorhydrate | | | | |
| and/or aluminum- | | | | |
| zirconium-glycine | | | | |
| compound | | | | |
| Alkyl(oligo)glucoside | 0.01-5 | 0.02-2.5 | 0.05-1 | 0.05-1 |
| with the INCI trade | | | | |
| name “Decyl Glucoside” | | | | |
| Di-n-octylether | 0.01-5 | 0.02-2.5 | 0.05-1 | 0.05-1 |
| Glycerolmonooleate | 0.01-5 | 0.02-2.5 | 0.05-1 | 0.05-1 |
| Emulsifier | 0.1-10 | 0.5-8 | 1-5 | 2-4 |
| Chelating agent | 0.01-2 | 0.02-1.5 | 0.05-1 | 0.1-0.5 |
| Water and/or ethanol | Ad 100 | Ad 100 | Ad 100 | Ad 100 |
| | SM 17 | SM 18 | SM 19 | SM 20 |
| Aluminum chlorhydrate | 5-25 | 6-20 | 7-15 | 8-14 |
| and/or aluminum | | | | |
| sesquichlorhydrate | | | | |
| and/or aluminum- | | | | |
| zirconium-glycine | | | | |
| compound | | | | |
| Alkyl(oligo)glucoside | 0.01-5 | 0.02-2.5 | 0.05-1 | 0.05-1 |
| with the INCI trade | | | | |
| name “DecylGlucoside” | | | | |
| Di-n-octylether | 0.01-5 | 0.02-2.5 | 0.05-1 | 0.05-1 |
| Glycerolmonooleate | 0.01-5 | 0.02-2.5 | 0.05-1 | 0.05-1 |
| Emulsifier^{a)} | 0.1-10 | 0.5-8 | 1-5 | 2-4 |
| EDTA | 0.01-2 | 0.02-1.5 | 0.05-1 | 0.1-0.5 |
| Water | Ad 100 | Ad 100 | Ad 100 | Ad 100 |
| | SM 21 | SM 22 | SM 23 | SM 24 |
| Aluminum chlorhydrate | 5-25 | 6-20 | 7-15 | 8-14 |
| and/or aluminum | | | | |
| sesquichlorhydrate | | | | |
| and/or aluminum- | | | | |
| zirconium-glycine | | | | |
| compound | | | | |
| Alkyl(oligo)glucoside | 0.01-5 | 0.02-2.5 | 0.05-1 | 0.05-1 |
| with the INCI trade | | | | |
| name “Decyl Glucoside” | | | | |
| Di-n-octylether | 0.01-5 | 0.02-2.5 | 0.05-1 | 0.05-1 |
| Glycerolmonooleate | 0.01-5 | 0.02-2.5 | 0.05-1 | 0.05-1 |
| Emulsifier^{a)} | 0.1-10 | 0.5-8 | 1-5 | 2-4 |
| EDTA | 0.01-2 | 0.02-1.5 | 0.05-1 | 0.1-0.5 |
| Ethanol | 10-60 | 20-50 | 30-40 | 30-40 |
| Water | Ad 100 | Ad 100 | Ad 100 | Ad 100 |

^{a)}The emulsifier is selected from ethoxylated C₁₄-C₂₀ alkanols having 2 to 30 mol of ethylene oxide per mol of alkanol and/or propoxylated C₁₄-C₂₀alkanols having 10 to 20 mol of propylene oxide per mol of alkanol, as well as the mixtures thereof

A further subject matter of the present invention is the use of a combination of
i. at least one alkyl(oligo)glucoside of the general formula RO-[G]ₓ, wherein R denotes an alkyl- and/or alkenyl radical having 4 to 22 carbon atoms, G denotes a sugar radical having 5 or 6 carbon atoms and x denotes integers from 1 to 10,
ii. t least one dialkyl ether with the same or different C4- to C14 alkyl radicals and
iii. at least one glycerine ester having a fatty acid selected from the group consisting of fatty acid monoglycerides, fatty acid diglycerides and mixtures thereof, alkyl(oligo)glucosides, selected dialkylethers and a glycerine ester in an antiperspirant aluminum salt and having an aqueous or aqueous-alcoholic carrier for reducing the sticky effect on the skin.

With respect to other preferred embodiments of the invention, the statements made about the antiperspirant agents apply mutatis mutandis.

A further subject matter of the present invention is a non-therapeutic cosmetic method for preventing and/or reducing the transpiration of the body, wherein an antiperspirant cosmetic agent according to the invention is applied to the skin, more particularly to the skin of the armpits, and remains on the skin for at least 1 hour, preferably for at least 2 hours, more preferably for at least 4 hours and most preferably for at least 6 hours.

The method using the antiperspirant agent according to the invention, which contains a combination of an antiperspirant aluminum salt in combination with a selected alkyl(oligo)glucoside, a selected dialkyl ether and a glycerine ester having a fatty acid, results in improved cosmetic properties, more particularly a reduced sticky effect.

With respect to the method, more particularly with respect to the cosmetic agent used therein, the statements made about the antiperspirant agents, as well as the use thereof, apply mutatis mutandis.

### EXAMPLES

1. Antiperspirant Roll-On—Emulsions

| | 1.1 | 1.2 | 1.3 | 1.4. | 1.5. | 1.6. |
|---|---|---|---|---|---|---|
| | wt. % | wt. % | wt. % | wt. % | wt. % | wt. % |
| PPG-15 Stearyl ether | 0.5 | 0.5 | 0.5 | 0.5 | 0.5 | 0.5 |
| Steareth-2 | 2.5 | 2.5 | 2.5 | 2.5 | 2.5 | 2.5 |
| Steareth-21 | 1.0 | 1.0 | 1.0 | 1.0 | 1.0 | 1.0 |
| Aluminum | — | — | — | — | 25.0 | — |
| sesquichlorohydrate | | | | | | |
| (Reach 301 L, 40% in | | | | | | |
| water) | | | | | | |
| Aluminum chlorohydrate | 27.0 | 27.0 | 27.0 | 27.0 | — | — |
| (Locron L from Clariant, | | | | | | |
| 50% in water) | | | | | | |
| Aluminum zirconium | — | — | — | — | — | 30.0 |
| tetrachlorohydrex | | | | | | |
| (AZG 364 from Summit | | | | | | |
| Reheis) | | | | | | |
| EDTA BX Powder | 0.1 | 0.1 | 0.1 | 0.1 | 0.1 | 0.1 |
| (BASF) | | | | | | |
| Decyl glucoside | 0.05 | 0.1 | 0.25 | 0.4 | 0.25 | 0.5 |
| Di-n-octylether | 0.05 | 0.1 | 0.5 | 0.5 | 0.25 | 0.5 |
| Glycerolmonooleate | 0.05 | 0.075 | 0.25 | 0.1 | 0.125 | 0.25 |
| Perfume | 1.0 | 1.0 | 1.0 | 1.0 | 1.0 | 1.0 |
| Water | Ad | Ad | Ad | Ad | Ad | Ad |
| | 100 | 100 | 100 | 100 | 100 | 100 |

2. Antiperspirant Roll-On—Alcoholic Solutions

| | 2.1 | 2.2 | 2.3 |
|---|---|---|---|
| | wt. % | wt. % | wt. % |
| Ethanol (96%) | 30.0 | 30.0 | 40.0 |
| Hydroxyethyl cellulose (Tylose H) | 0.4 | 0.4 | 0.4 |
| Ceteareth-12 (Eumulgin B1, BASF) | 2.0 | 2.0 | 2.0 |
| Ceteareth-30 (Eumulgin B3, BASF) | 2.0 | 2.0 | 2.0 |
| Phenoxyethanol | 0.5 | 0.5 | 0.5 |
| Aluminum chlorohydrate (Locron L | 16.0 | 16.0 | — |
| from Clariant, 50% in water) | | | |
| Aluminum zirconium tetrachlorohydrex | — | — | 20 |
| (AZG 364 from Summit Reheis) | | | |
| EDTA BX Powder (BASF) | 0.1 | 0.1 | 0.1 |
| Decyl glucoside | 0.05 | 0.25 | 0.1 |
| Di-n-octylether | 0.05 | 0.5 | 0.1 |
| Glycerolmonooleate | 0.05 | 0.25 | 0.075 |
| Citric acid triethyl ester | 1.0 | 3.0 | 0.5 |
| Perfume | 1.0 | 1.0 | 1.0 |
| Water | Ad 100 | Ad 100 | Ad 100 |

1. Antiperspirant agent comprising, in an aqueous or aqueous-alcoholic carrier,
a) at least one antiperspirant aluminum salt, selected from the group of water-soluble inorganic aluminum salts, water-soluble inorganic aluminum salts, water-soluble inorganic aluminum zirconium salts, water-soluble organic aluminum zirconium salts and mixtures thereof,
b) at least one alkyl(oligo)glucoside of the general formula RO-[G]ₓ, wherein R denotes an alkyl- and/or alkenyl radical having from 4 to 22 carbon atoms, G denotes a sugar radical having 5 or 6 carbon atoms and x denotes integers from 1 to 10,
c) at least one dialkyl ether with the same or different C4- to C14 alkyl radicals and
d) at least one glycerine ester having a fatty acid, selected from the group of fatty acid monoglycerides, fatty acid diglycerides and the mixtures thereof.

2. Antiperspirant cosmetic agent according to claim 1, wherein at least one antiperspirant aluminum salt is selected from the group of aluminum chlorhydrate, aluminum sesquichlorhydrate, aluminum zirconium tetrachlorhydrexglycine and mixtures thereof.

3. Antiperspirant cosmetic agent according to claim 1, wherein at least one antiperspirant aluminum salt is included in a quantity from about 5 to about 25 wt. %, each of the quantity values referring to the total weight of the antiperspirant agent.

4. Antiperspirant cosmetic agent according to claim 1, wherein the at least one alkyl(oligo)glucoside comprises an alkyl(oligo)glucoside, which is known under the INCI designation Decyl Glucoside.

5. Antiperspirant cosmetic agent according to claim 1, wherein the at least one alkyl(oligo)glucoside is an alkyl(oligo)glucoside, which is known under the INCI designation Decyl Glucoside, and that this is included in a quantity from about 0.05 to about 1 wt. %, relative to the total agent.

6. Antiperspirant cosmetic agent according to claim 1, wherein the antiperspirant cosmetic agent comprises at least one dialkylether di-n-octylether.

7. Antiperspirant cosmetic agent according to claim 1, wherein the at least one dialkylether is di-n-octylether in a quantity from about 0.05 to about 1 wt. %, relative to the total agent.

8. Antiperspirant cosmetic agent according to claim 1, wherein the at least one glycerine ester having a fatty acid is glycerolmonooleate in a quantity from about 0.05 to about 1 wt. %, relative to the total agent.

9. Non-therapeutic cosmetic method for preventing and/or reducing the transpiration of the body, the method comprising the steps of applying an antiperspirant agent according to claim 1 to skin, and allowing the antiperspirant agent to remain on the skin for at least about 1 hour.

10. A method comprising the step of using of a combination of
i. at least one alkyl(oligo)glucoside of the general formula RO-[G]ₓ, wherein R denotes an alkyl- and/or alkenyl radical having from 4 to 22 carbon atoms, G denotes a sugar radical having 5 or 6 carbon atoms and x denotes integers from 1 to 10,
ii. at least one dialkyl ether with the same or different C4- to C14 alkyl radicals and
iii. at least one glycerine ester having a fatty acid selected from the group of fatty acid monoglycerides, fatty acid diglycerides and mixtures thereof, alkyl(oligo)glucosides, selected dialkylethers and a glycerine ester in an antiperspirant aluminum salt and having an aqueous or aqueous-alcoholic carrier for reducing the sticky effect on the skin.

11. Antiperspirant cosmetic agent according to claim 1, wherein at least one antiperspirant aluminum salt is included in a quantity from about 6 to about 20 wt. %, each of the quantity values referring to the total weight of the antiperspirant agent.

12. Antiperspirant cosmetic agent according to claim 1, wherein at least one antiperspirant aluminum salt is included in a quantity from about 7 to about 15 wt. %, each of the quantity values referring to the total weight of the antiperspirant agent.

13. Antiperspirant cosmetic agent according to claim 1, wherein at least one antiperspirant aluminum salt is included in a quantity from about 8 to about 14 wt. %, each of the quantity values referring to the total weight of the antiperspirant agent.

14. The method according to claim 9, wherein allowing comprises allowing the antiperspirant agent to remains on the skin for at least about 2 hours.

15. The method according to claim 9, wherein allowing comprises allowing the antiperspirant agent to remain on the skin for at least about 4 hours.

16. The method according to claim 9, wherein allowing comprises allowing the antiperspirant agent to remain on the skin for at least about 6 hours.
